(19) United States
(12) Patent Application Publication
(10) Pub. No.: US 2013/0011450 A1
(43) Pub. Date: Jan. 10, 2013

(54) DEODORIZING FIBER STRUCTURE

(75) Inventors: Rumi Karasawa, Otsu (JP); Chika Kimura, Otsu (JP); Masami Ikeyama, Otsu (JP); Takahiro Ono, Otsu (JP); Keiji Takeda, Otsu (JP)

(73) Assignee: TORAY INDUSTRIES, INC., Tokyo (JP)

(21) Appl. No.: 13/636,518

(22) PCT Filed: Mar. 25, 2011
(86) PCT No.: PCT/JP2011/057289

§ 371 Date: Sep. 21, 2012

(30) Foreign Application Priority Data
Mar. 25, 2010 (JP) 2010-069769

(51) Int. Cl. A61L 9/012; A61L 9/014; A61L 9/01

(52) U.S. Cl. 424/400; 424/76.1

(57) ABSTRACT

A deodorizing fiber structure contains a material composed of a hydroxy acid derivative is secured to a polyester-based fiber structure, and a method for producing a fiber structure including the steps of immersing a polyester-based fiber structure in a hydroxy acid aqueous solution, subsequently drying, and then heat-treating.

### RELATED APPLICATIONS

This is a §371 of International Application No. PCT/JP2011/057289, with an international filing date of Mar. 25, 2011 (WO 201 1/1 18749 A1, published Sep. 29, 2011, which is based on Japanese Patent Application No. 2010-069769 filed Mar. 25, 2010, the subject matter of which is incorporated herein by reference.

### TECHNICAL FIELD

This disclosure relates to a deodorizing polyester-based fiber structure excellent in Washing durability.

### BACKGROUND

In recent years, with diversification of life styles, the awareness of health and sanitation issues rises, and in the respective fields of food, clothing and shelter, products with deodorizing and antimicrobial functions are being practically used. In particular in view of health promotion, various indoor and outdoor exercises are practiced actively, and there is a growing demand for textile products highly capable of absorbing and deodorizing large volumes of perspiration produced from exercising. Further, in aging-related care and medical fields, diverse functions including not only deodorization but also water absorption and water repellency are required to be provided for optimization purposes, and there is a high demand for products having sophisticated deodorizing functions.

As methods for giving deodorizing capability, proposed are a method of using a metal complex such as a Metal phthalocyanine (JP 64-20852 A), a method of depositing a deodorizing extract from a plant or the like onto fibers (JP 9-271484 A), a method of using a polycarboxylic acid resin and a photocatalyst (JP 2004-052208 A), etc. However, these methods are low in washing durability, and there is a problem that if the amounts of the deodorizing agent and the binder used are increased to enhance the deodorizing capability still after washing, appearance quality such as hand is impaired.

It could therefore be helpful to provide a polyester-based fiber structure having all of high deodorizing capacity, advanced deodorizing capability excellent in washing durability, and good texture.

### SUMMARY

We thus provide:

(1) A deodorizing fiber structure in which a material composed of a hydroxy acid derivative is secured to a polyester-based fiber structure.

(2) A deodorizing fiber structure, according to the abovementioned (1), wherein the material composed of said hydroxy acid derivative is a material composed of any one of the monomer, polymer or copolymer of the hydroxy acid derivative.

(3) A deodorizing fiber structure, according to the abovementioned (1) or (2), wherein the material composed of said hydroxy acid derivative is a derivative of at least one compound selected from citric acid, malic acid and tartaric acid.

(4) A deodorizing fiber structure, according to any one of the abovementioned (1) through (3), wherein the material composed of said hydroxy acid derivative is a derivative of citric acid.

(5) A deodorizing fiber structure, according to any one of the abovementioned (1) through (4), wherein said polyester-based fiber structure contains a pyridine-based antimicrobial agent.

(6) A deodorizing fiber structure, according to any one of the abovementioned (1) through (5), wherein a water absorbing agent is deposited on said polyester-based fiber structure.

(7) A deodorizing fiber structure, according to the abovementioned (6), wherein said water absorbing agent is a hydrophilic polyester-based resin.

(8) A deodorizing fiber structure, according to any one of the abovementioned (1) through (5), wherein a water repellent is deposited on said polyester-based fiber structure.

(9) A method for producing a fiber structure comprising the steps of immersing a polyester-based fiber structure in a hydroxy acid aqueous solution, subsequently drying, and then heat-treating.

Our polyester-based fiber structures thus have all of high deodorizing capacity, advanced deodorizing capability excellent in washing durability, and good texture.

### DETAILED DESCRIPTION

We considered the problem of providing high deodorizing capacity, advanced deodorizing capability excellent in washing durability, and good texture to a polyester-based fiber structure and, as a result, discovered that we can, all at once, secure a material composed of a hydroxy acid derivative to a polyester-based fiber structure.

We found that if a polyester-based fiber structure is immersed in a hydroxy acid aqueous solution and/or a hydroxy acid salt aqueous solution, being followed by heat treatment, the hydroxy acid or hydroxy acid salt deposited on the polyester-based fiber structure chemically reacts to produce a hydroxy acid derivative, thereby having the monomer, polymer or copolymer of the hydroxy acid secured to the polyester-based fiber structure. The mode of the chemical reaction of the hydroxy acid is not clear, but we believe that the hydroxy group and the carboxyl group of the hydroxy acid react under heating to cause polymerization, hence hydrophobization and, therefore, the material composed of the hydroxy acid derivative is strongly deposited or secured to the surfaces of the polyester-based fibers with high affinity, or that the hydroxy group and the carboxyl group of the hydroxy acid react with or are interesterified with some hydroxy groups and carboxyl groups existing at the ends of polyester-based fibers, to he secured to the polyester-based fibers, thereby obtaining very high durability.

The “securing” includes a case where the abovementioned hydropbobized polymer is secured to the surfaces of polyester-based fibers with high affinity, a case where the hydroxy acid is secured, for example, by reaction with the hydroxy groups and carboxyl groups existing at the ends of fibers, and further a case where the hydroxy acid adheres to the surfaces of fibers and also a case where the hydroxy acid permeates inside the fibers. The adhesion includes a state where the hydroxy acid and the surfaces of fibers physically adhere to or are chemically bonded to each other.

Owing to the strong securing, the deodorizing capability barely declines after 10 times or 50 times of household washing or after industrial washing, and the fiber structure obtained can conform to the Textile Product Deodorization Process Certification Standard of Fiber Evaluation Technical Council (in Japanese in which the ammonia deodorizing capability after 10 times of washing should be 70% or more. The deposition strength can be seen also from the excellent washing durability that the ammonia deodorizing capability after 50 times of washing is 60% or more.

As examples of the hydroxy acid, enumerated are glycollic acid, lactic acid, tartronic acid, glyceric acid, hydroxybutyric acid, mallic acid, citric acid, tartaric acid, citramalic acid, isocitric acid, leucic acid, inevalonic acid, pantoic acid, ricinoleic acid, ricinoelaidic acid, cerebronic acid, quinic acid, shikimic, acid, salicylic acid, creosotic acid, vanillic acid, syringic acid, pyrocatechuic acid, resorcylic acid, protocatechuic acid, gentisic acid, orsellinic acid, gallic acid, mandelic acid, beazilic acid, atrolactinic acid, melilotic acid, phloretic acid, coumaric acid, umbellic acid, caffeic acid, ferulic acid, sinapic acid, etc. Citric acid, alit acid and tartaric acid are preferred in view of high safety and easy availability as can be seen from the fact that they are also used for edible products. Citric acid is more preferred since the number of carboxyl groups per molecule is large.

It is preferred that the deposited amount of the hydroxy acid derivative per 100 parts by weight of the polyester-based fiber structure is 0.01 to 100 parts by weight. A more preferred range is 0.1 to 10 parts by weight. if the deposited amount is smaller than 0.01 part by weight, sufficient deodorization performance may not be obtained as the case may be. Further, it is not preferred in view of cost that the amount is larger than 100 parts by weight, since the amount of the hydroxy acid not secured increases. In addition in this case, the fastness tends to decline, and the texture tends to be hard.

There is no particular limit to the method of immersing the polyester-based fiber structure in a hydroxy acid and/or hydroxy acid salt aqueous solution, but a general method such as pad treatment, bath treatment or coating treatment can be used.

In case of pad treatment, a polyester-based fiber structure is immersed in a hydroxy acid and/or hydroxy acid salt aqueous solution, squeezed by a mangle, dried, and preferably treated by dry heat or wet heat at a temperature of 70 to 200° C. for 0.1 to 30 minutes. Dry heat treatment is preferred since good adhesion can be obtained. A dry heat treatment at a temperature of 100 to 190° C. is more preferred. it is preferred to wash with water after completion of dry heat treatment or wet heat treatment.

In case of bath treatment, a dye and a hydroxy acid and/or a hydroxy acid salt can be placed in the same bath, or after completion of dyeing, a polyester-based fiber structure can be immersed in a hydroxy acid aqueous solution. It is preferred that a polyester-based fiber structure is immersed in a hydroxy acid and/or hydroxy acid salt aqueous solution and heat-treated preferably at a temperature of 100 to 140° C. for 5 to 60 minutes. Further, after completion of heat treatment, it is preferred to wash with water.

The concentration of the hydroxy acid and/or hydroxy acid salt aqueous solution can be adjusted as appropriate to ensure that the deposited amount of the hydroxy acid derivative in the finally obtained fiber structure may be kept in a preferred range. For example, approx. 5 g/L to approx. 200 g/L is preferred.

General function-imparting agents can also be added to the deodorizing fiber structure.

It is preferred that the fiber structure contains a pyridine-based antimicrobial agent. The pyridine-based antimicrobial agent is not especially limited. For example, usable are nitrile-based compounds such as 5-chloro-2,4,6-trifluoroisophthalonitrile, pyridine-based compounds such as 2-chloro-6-trichloromethylpyridine, 2-chloro-4-trichioromethyl-6-metboxypyridine, 2-chloro-4-trichloromehyl-642-furylmethoxy)pyridine, di(4-chlorophenyppyridylmethanol, 2,3,5 -trichloro-4-(n-propylsulfonyl)pyridine, 2-pyridylthio1-1 oxide zinc, and di(2-pyridylthiol-1-oxide), haloalkylthio-based compounds such as N-trichloromethy thiophthalimide, N-1,1,2,2- tetrachloroethylthiotetrahydrophthalimide, N-trichloromethylthiatetrahydrophthalimide, N-trichloromethythio-N-(phenyl)methylsulfamide, N-triehloromethylthio-N-(4-chlorophenyl)methylsulfamide, N-(1-fluoro-1,1,2.2 -tetrachloroethylthio)-N-(Phenyl)methylsulfamide, N-(1,1-difluoro-1,2,2-trichloroethylthio)-N-(phenyl)methylsulfamide, N,N-dichlorofluoromethylthio-N′-phenylstilfarnide, and N,N-dimethyl-N-(p-tolyl)-N′(fluorodichloromethylthio)sulfamide, organic iodo-based compounds such as 1-diiodomethylsulfonyl-4-chlorobenzene, 3-iodo-2-propargylbutylcarbmic acid, 4-chlorophenyl-3-iodopropargylformal, 3-ethoxycarbonyloxy-1-brom-1,2-diiodo-1-propene, and 2,3,3-triiodoallylalcohol, thiazole-based compounds such as 4,5-dichloro-2-cyclohexyl-4-isothiazoline-3-one, 2-(4-thiocyanomethylthio)benzthiazole, and 2-mercaptobenzthiazole zinc, benzimidazole-based compounds such as 1H-2-thiocyanomethylthiobenzimidazole, and 2-(2-chlorophenyl)-1H-benzimidazole, etc.

Among them, to obtain both high washing durability and the deodorizing performance by the hydroxy acid derivative, an antimicrobial agent with a specific molecular weight, a specific inorganicity/organicity value and a specific average particle size is preferred. As the antimicrobial agent, a specific antimicrobial agent having a molecular weight of preferably 200 to 700, more preferably 300 to 500, an inorganicity/organicity value of 0.3 to 2.0, and an average particle size of preferably 2 μm or smaller, more preferably 1 μm or smaller is used.

If the molecular weight is lower than 200, the antimicrobial agent can be deposited or exhausted/diffused in the polyester-based fibers, but is low in washing durability. On the other hand, if the molecular weight is higher than 700, the antimicrobial agent cannot be deposited or exhausted in the polyester fibers. A preferred range of the molecular weight of the antimicrobial agent is 300 to 500.

The abovementioned “inorganicityiorganicity value” refers to the idea contrived by Mr. Minoru Fujita for handling the polarity values of various organic compounds in terms of organicity concept (see Chemical Experiment Science, Organic Chemistry, Revised Edition, Kawade Shobo (1971)(in Japanese)). According to this idea, one carbon atom (C) is defined, to have an organicity value of 20 and, in relation with it, the values of inorganicity and organicity of various polar groups are defined as shown in Table 1 of that book. For each compound, the sum of inorganicity values and the sum of organicity values are obtained, and the ratio of both the sums is obtained as the value for the compound.

The inorganicity/organicity value of, for example, polyethylene terephthalate calculated according to this organicity concept is 0.7. In this case, attention is paid to the affinity between synthetic fibers and an antimicrobial agent on the basis of the value calculated according to the organicity concept, and the antimicrobial agent with the inorganicity/organicity value in a predetermined, range is deposited or exhausted/diffused into polyester-based fibers,

If the inorganicity/organicity value is smaller than 0.3, the organicity is too strong, and if the value is larger than 1.4 on the contrary, the inorganicity is too strong, and the antimicrobial agent is hard to be deposited or exhausted/diffused into polyester-based fibers. It is preferred that the inorganicity/organicity value is from 0.35 to 1.3, and a more preferred range is 0.4 to 1.2.

The antimicrobial agent can be given to the fiber structure before or after or as soon as the hydroxy acid is secured to the fiber structure. If both a hydroxy acid and a pyridine-based antimicrobial agent are secured to polyester-based fibers, both the substances show high washing durability, and both deodorizing performance and antimicrobial performance can be assured.

Further, it is preferred that a water absorbing agent is deposited on the surfaces of the fibers. There is no particular limit to the water absorbing agent used, but an ordinary water absorbing agent such as a polyester-based resin or silicone-based resin can be used. Above all, a hydrophilic polyester-based resin is preferred, and as the hydrophilic polyester-based resin, a polyester ether copolymer obtained by copolymerizing polyethylene glycol to a polyester segment consisting of an acid component and a glycol component can he preferably used. The acid component can be at least one component selected from dimethyl terephthalate, dimethyl isophthalate, 5-sodiumsulfoisophthalic acid, terephthalic acid, isophthalic acid, adipic acid, etc. The glycol component can be at least one component selected from ethylene glycol, 1,2-proptmediol, 1,3-propanediol, 1,2-butanediol, 1,4-butanediol, 1,6-hexanediol and diethylene glycol. As the molecular weight of the polyethylene glycol, a range from 800 to 3000 can be preferably used. Specifically, a copolymerized polyester with the molar ratio of dimethyl terephthalatelethylene glycol in a range from 7/3 to 9/1, with the number of repeating units in a range from 5 to 8, and with the molecular weight of polyethylene glycol in a range from 8000 to 30000, and a copolymerized polyester resin consisting of 250/200/330 parts of a reaction mixture consisting of dimethyl terephthalate/dimethy 5-sodiumsulfoisophthalatelethylene glycol, and 100 parts of polyethylene glycol with a molecular weight of 2000, can be presented as examples.

The methods for giving a hydrophilic polyester-based resin to a fiber structure include a method of giving a hydroxy acid and/or hydroxy acid salt aqueous solution to a fiber structure, to produce a hydroxy acid derivative and subsequently giving a hydrophilic polyester-based resin, a method of giving a mixture consisting of a hydrophilic polyester-based resin and a hydroxy acid and/or hydroxy acid salt to a fiber structure, a method of giving a hydrophilie polyester-based resin to a fiber structure and subsequently giving a hydroxy acid and/or hydroxy acid salt, and so on. Among the methods, a method of giving a hydrophilic polyester-based resin to a fiber structure and subsequently securing a hydroxy acid derivative is more preferred for the reason that if the hydroxy acid derivative exists on the outermost surface, offensive odor is likely to contact the hydroxy acid derivative, thereby assuring, advanced deodorizing capability.

In the case where a mixture consisting of a hydroxy acid and a hydrophilic polyester-based resin is provided on a fiber structure, the mixing ratio as a ratio by weight of the solid content of the hydroxy acid derivative to the solid content of the polyester-based resin is hydroxy acid derivative solid content/polyester-based resin solid content=100/0 to 100/100. A preferred range is 100/0 to 100/40.

Further, it is preferred that the fiber structure has a water repellent deposited on the surfaces of the fibers. There is no particular limit to the water repellent used and an ordinary water repellent such as a silicone-based water repellent, fluorine-based water repellent or paraffin-based water repellent can be used. In view of durability, a fluorine-based water repellent is preferred. Further, in view of higher durability, a melamine resin, or a polyfunctional blocked isocyanate group-containing urethane resin can also be added to the water repellent for use in combination, it is preferred that the water repellent is given basically together with the hydroxy acid derivative or after the hydroxy acid derivative is secured.

Furthermore, function-imparting agents include an inorganic deodorizer, neutral or basic organic deodorizer, photocatalyst, stain-proofing agent, moisture absorbing agent, antistatic agent, colorant, anti-slip agent and the like.

There is no particular limit to the polyester-based fiber structure. Examples of the fibers include aromatic polyester-based fibers of polyethylene terephthalate, polypropylene terephthalate, polybutylene terephthalate or the like, fibers composed of eopoymers using, for example, isophthalic acid, isophthalie acid sulfanate, adipic acid or the like as the acid component or alcohol component of an aromatic polyester, aromatic polyester-based fibers blended with polyethylene glycol or the like, aliphatic polyester-based fibers typified by those containing L-lactic acid as a main component, etc. Any one type of these fibers can be used alone or two or more types of these fibers can also be used as a mixture,

Further, the fibers can be ordinary flat yarns, or also other fiat yams such as false-twisted yarns, strong twisted yarns, Taslan yarns, slub yarns and blended yams, or various other modes of yarns such as staple fibers, tows and spun yarns.

The fiber structure can be a fabric such as knitted fabric, woven fabric or nonwoven fabric or cords respectively formed of the aforementioned fibers, and so on.

The fiber structure has durability and deodorizing capability and therefore can be suitably used for clothing and bedding, specifically, sports shirts, school uniforms, care clothes, white robes and gowns, blouses, dress shirts, skirts, slacks, coats, blousons, windbreakers, gloves, hats, mattress sheets, mattress covers, curtains, tents, etc. for clothing applications and non-clothing applications.

### EXAMPLES

The fiber structure is explained below in detail in reference to examples, but is not limited thereto or thereby. The qualities of the examples were evaluated according to the following methods.

### (Washing Method)

As specified in Attached Table 1-103 of JIS L 0217 “Labeling Marks for Handling of Textile Products and Labeling Methods Thereof” (1995), water of 40±2° was placed in a home use electric washing machine to achieve a bath ratio of 1:30, and a weakly alkaline synthetic detergent was added to be dissolved for washing under the strong condition for 5 minutes, followed by draining/dewatering, washing with water for 2 minutes, dewatering, washing with water for 2 minutes again, and dewatering. This process as one cycle was repeated 10 times or 50 times, and the washed sample was hung to be dried and evaluated,

### (Industrial Washing Method)

Water of 60±2° C. was placed in a drum type washer/dryer (WT946wps produced by Miere) to achieve a bath ratio of 1:10, and 2 g/L of phosphorus-free detergent Dash (produced by Lion Hygiene Corporation) and 2 g/L, of sodium metasilicate were added to be dissolved for washing for 45 minutes, followed by draining/dewatering, washing with water of 40° C. for 9 minutes, dewatering, washing with water again for 5 minutes, dewatering, and drying at 100° C. for 46 minutes. This process as one cycle was repeated 15 times, for evaluation.

### (Deodorizing Capability)

A 500 ml container containing a cut sample with a size of 10 cm×5 cm was charged with ammonia gas to achieve an initial concentration of 300 ppm and was closed tightly, being allowed to stand for 30 minutes, and subsequently a gas detector tube was used to measure the remaining ammonia concentration. A similar test was made without using any sample, and the remaining ammonia concentration was measured as a blank test concentration. The offensive odor rate (%) was calculated from the following formula.

Offensive odor rate (%)=(1−(Gas detector tube measured concentration)/(Blank test concentration))×100

A larger value shows a higher deodorizing capability.

### (Antimicrobial Capability)

The antimicrobial capability against Klebsiella pneumoniae was evaluated according to JIS L 1902 “Quantitative Test (Absorption Method) according to Testing for Antibacterial Activity and Efficacy on Textile Products”.

0≦L (microbicidal activity value) is acceptable.

### (Water Absorbability)

A water drop was dropped on a fabric by the method specified in JIS L 1096, and the time taken for the water to be completely absorbed was measured and indicated in seconds.

### (Water Repellency)

Evaluation was made by the spray method according to JIS L 1092 “Testing Methods for Water Resistance of Textiles” (1998), to decide the class.

### (Examples 1 and 2)

Polyethylene terephthalate yarns of 84 decitexes and 72 filaments and polyethylene terephthalate yarns of 84 decitexes and 36 filaments were used to form a knitted fabric, and the knitted fabric was scoured, dried and intermediately set according to conventional methods. Then, it was dyed according to a conventional using a jet dyeing machine, washed with hot water and dried. The fabric was immersed in either of the following hydroxy acid aqueous solutions, squeezed by a mangle to achieve a squeezing rate of 91%, dried at 130° C., and set at 170° C. for 1 minute.

### Example 1

### Citric Acid (Anhydrous) (Nacalai Standard Class 1, produced by NACALAI TESQUE, INC.) 18 g/L

### Example 2

### Citric Acid (Anhydrous) (Nacalai Standard Class 1, produced by NACALAI TESQUE, INC.) 100 g/L

The obtained flibric was excellent in deodorizing capability and washing resistance as shown in Table 1.

### Example 3

The same knitted fabric as that used in Example 1 was treated as described in Example 1, except that it was subsequently washed with hot water at 60° C., followed by washing with water, dewatering, drying, and setting at 150° C. for 1 minute for finishing, to obtain a fabric of Example 3. The obtained fabric was excellent in deodorizing capability and washing resistance as shown in Table 1.

### Examples 4, 5 and 6

The same knitted fabric as the fabric used in Example 1 was treated as described in Example 1, except that it was immersed in any of the following hydroxy acid aqueous solutions, to obtain the fabrics of Examples 4, 5 and 6. The obtained fabrics were excellent in deodorizing capability and washing resistance as shown in Table 1.

### Example 4

### DL Malic Acid (Nacalai Standard Class 1, produced by NACALAI TESQUE, INC.), 30 g/L

### Example 5

### L-(+)-Tartaric Acid (Nacalai Standard Class 1, produced by NACALAI TESQUE, INC.), 30 g/L

### Example 6

### Lactic Acid (Nacalai Standard Class 1, produced by NACALAI TESQUE, INC.), 30 g/L

### Comparative Example 1

The knitted fabric used in Example l was dyed, washed with hot water and dried, but was not subsequently treated by a deodorizing agent aqueous solution. The performance of the fabric was evaluated as described in Example 1. The results are shown in Table 1.

### Comparative Examples 2 to 4

The same knitted fabric as that used in Example l was treated as described in Example 1, except that it was immersed in any of the following chemical aqueous solutions, to obtain the fabrics of Comparative Examples 2 to 4. The obtained fabrics were inferior especially in washing resistance as shown in Table 1.

### Comparative Example 2

### Adipic Acid (Nacalai Standard Class 1, produced by NACALAI TESQUE, INC.), 30 g/L

### Comparative Example 3

### Malonic Acid (Nacalai Standard Class 1, produced by NACALAI TESQUE, INC.), 30 g/L

### Comparative Example 4

### Polyarrylic Acid Resin (Aqualiel-IL415 Produced by Nippon Shokubai Co., Ltd.) (Solid Content 45%), 40 g/L

### Comparative Example 5

A woven fabric of cotton 100% (shirting, No. 3) was used as Comparative Example 5, and the deodorizing capabilities before and after washing were evaluated. The results are shown in Table 1.

### Comparative Example 6

The woven fabric of cotton 100% (shirting, No. 3) used in Comparative Example 5 was immersed in the hydroxy acid aqueous solution stated in Table 1, squeezed by a mangle to achieve a squeezing rate of 60%, dried at 130° C., and subsequently set at 170° C. for 1 minute. The obtained fabric was inferior in washing resistance as shown in Table 1.

### Example 7

Polyethylene terephthalate yarns of 72 decitexes and 60 filaments were used as warp threads and polyethylene terephthalate yarns of 56 decitexes and 24 filaments were used as weft threads to weave a twill weave fabric at a warp density of 118 threads/2.54 cm and at a weft density of 70 threads/2.54 cm, and the fabric was scoured, dried and intermediately set according to conventional methods. Then, the fabric was immersed in a solution having the following hydroxy acid and antistatic agent dissolved/dispersed therein, squeezed by a mangle to achieve a squeezing rate of 53%, dried at 130° C., and subsequently set at 170° C. for 1 minute. Then, the fabric was washed with hot water at 60° C., followed by washing with water, dewatering, drying and subsequently setting at 150° C. for 1 minute for finishing,

The obtained fabric was excellent in the washing resistance of deodorizing capability and antimicrobial capability as shown in Table 2.

### Citric Acid (Anhydrous) (Nacaiai Standard Class 1, Produced by NACALAI TESQUE, INC.), 18 g/L

### “MR-T100” (Pyridine-Based Antistatic Agent, Solid Content 19%, Produced by Osaka Kasei Co., Ltd:), 15 g/L

### Example 8

The same woven fabric as that of Example 7 was immersed in an aqueous dispersion of the following antimicrobial agent, squeezed by a mangle to achieve a squeezing rate of 53%, dried at 130° C., and subsequently set at 170° C. for 1 minute.

### “MR-T100” (Solid Content 19%, Produced by Osaka Kasei Co., Ltd.), 15 g/L

Then, the obtained woven fabric was immersed in the following hydroxy acid aqueous solution, squeezed by a mangle to achieve a squeezing rate of 55%, dried at 130° C., subsequently set at 1.70° C. for 1 minute, then washed with hot water at 60° C., followed by washing with water, dewatering, drying and then setting at 150° C. for 1 minute for finishing.

Citric Acid (Anhydrous) (Nacalai Standard class 1, Produced by NACALAI TESQUE,INC). 18 g/L

### Example 9

The same woven fabric as that of Example 7 was scoured, dried and intermediately set according to conventional methods. Then, it was immersed in a solution of a water absorbing agent {hydrophilic polyester-based resin: “TM-SS21” (produced by Matsumoto Yushi-Seiyaku Co., Ltd.) 6% owf, bath ratio 1:10, pH 5)} using a jet dyeing machine, and treated at 130° C. for 60 minutes according to a conventional dyeing method. The treated fabric was immersed in the following hydroxy acid aqueous solution, squeezed by a mangle to achieve a squeezing rate of 53%, dried at 130° C., and subsequently set at 170° C. for 1 minute, and the obtained fabric was excellent in deodorizing capability, washing resistance and water absorbability as shown in Table 2.

### Citric Acid (Anhydrous) (Nacalai Standard Class 1, Produced by NACALAI TESQUE, INC.), 18 g/L

### Example 10

The same woven fabric as the fabric used in Example 7 was treated as described in Example 9 by immersing in the water absorbing agent solution and then in the hydroxy acid aqueous solution, and subsequently washed with hot water and set at 150° C. for I minute for finishing. The obtained fabric was excellent in deodorizing capability, washing resistance and water absorbability as shown in Table 2.

### Example 11

The same woven fabric as that of Example 7 was scoured, dried and intermediately set according to conventional methods. Then, it was dyed according to a conventional method using a jet dyeing machine. It was immersed in ati aqueous solution containing 18 g/L of citric acid (anhydrous) (Nacalai Standard Class 1, produced by NACALAI TESQUE, INC.), squeezed by a mangle to achieve a squeezing rate of 53%, dried at 130° C., and subsequently set at 170° C. for 1 minute. Further, it was immersed in a function-imparting solution containing the following water absorbing components, squeezed by a mangle to achieve a squeezing rate of 53%, dried at 130° C., and subsequently set at 170° C. for 1 minute. The obtained fabric was excellent in deodorizing capability, washing resistance and water absorbability as shown in Table 2.

Water absorbing components:

(a) “SR1800” (hydrophilic polyester-based water absorbing agent, produced by Takamatsu Oil & Fat Co., Ltd.): 60 g/L
(b) “SR-CA-1” (catalyst for water absorbing agent, produced by Takamatsu Oil & Fat Co., ltd.): 6 g/L

### Example 12

The same woven fabric as the fabric used in Example 7 was immersed in an aqueous solution containing 18 g/L of citric acid (anhydrous) (Nacalai Standard Class 1, produced by NACALAI TESQUL, INC.), squeezed by a mangle to achieve a squeezing rate of 53%, dried at 130° C., subsequently set at 170° C. for 1 minute. Then, it was washed with hot water, dried at 130° C., subsequently immersed in a function-imparting solution containing the following water absorbing components, squeezed by a mangle to achieve a squeezing rate of 53%, dried at 130° C., and subsequently set at 170° C. for 1 minute. The obtained fabric was excellent in deodorizing capability, water resistance and water absorbability as shown in Table 2.

### Example 13

The same woven fabric as that of Example 7 was scoured, dried and intermediately set according to conventional methods. Then, it was dyed according to a conventional method using a jet dyeing machine. It was immersed in at function-imparting solution containing the following components, squeezed by a mangle to achieve a squeezing rate of 53%, dried at 130° C., and subsequently set at 170° C. for 1 minute. The obtained fabric was excellent in deodorizing capability, washing resistance and water absorbability as shown in Table 2.

Function-imparting components:

(a) Citric acid (anhydrous) (Nacalai Standard Class 1, produced by NACALAI TESQUE, INC.): 18 g/L
(b) “SR1800” (hydrophilic polyester-based water absorbing agent produced by Takamatsu Oil & Fat Co., Ltd.): 60 g/L
(c) “SR-CA-1” (catalyst for water absorbing agent, produced by Takamatsu it & Fat Co., Ltd.): 6 g/L

### Example 14

After the fabric was treated as described in Example 13, it was washed with hot water and set at 150° C. for 1 minute for finishing. The obtained fabric was excellent in deodorizing capability, washing resistance and water absorbability as shown in Table 2.

### Example 15

The same woven fabric as that of Example 7 was scoured, dried and intermediately set according to conventional methods. Then, it was dyed according to a conventional method using a jet dyeing machine. It was immersed in an aqueous solution containing 18 g/L of citric acid (anhydrous) (Nacalai Standard Class 1, produced by NACALAI TESQUE, INC.), squeezed by a mangle to achieve a squeezing rate of 53%, dried at 130° C., and subsequently set at 170° C. for 1 minute. Further, it was immersed in a function-imparting solution containing the following water repellent and crosslinking agent, squeezed by a mangle to achieve a squeezing rate of 53%, dried at 130° C., and subsequently set at 170° C. for 1 minute. The obtained fabric was excellent in deodorizing capability, washing resistance and water repellency as shown in Table 2.

Function-imparting components:

(a) “FX860” (fluorine-based water repellent oil produced by K.K. Kyokerikasei): 60 g/L
(b) “Beckamin M-3” (triazine ring-containing compound produced by Dainippon Ink and Chemicals, Inc.): 3 g/L
(c) “Beckamin ACX” (catalyst produced by Dainippon Ink and Chemicals, inc): 1 g/L

### Comparative Example 7

The same woven fabric as the fabric used in Example 7 was immersed in a solution containing 10 g/L of “ELENITE 139” (produced by Takamatsu Oil & Fat Co., Ltd.) as a temporary zintistutic agent, squeezed by a mange, dried at 130° C., and subsequently set at 160° C. for 1 minute. The obtained fabric did not have deodorizing capability and was inferior in water absorbability after washing as shown in Table 2.

TABLE 1

| | | | Ammonia deodorizing capability (%) | | | |
|---|---|---|---|---|---|---|
| | | | | | | After |
| | | Concen- | Before | After 10 times | After 50 times | industrial |
| | Function-imparting agent | tration | washing | of washing | of washing | washing |
| Example 1 | Citric acid (anhydrous) (Nacalai Standard Class 1, | 18 g/L | 100 | 88 | 81 | 80 |
| | produced by NACALAI TESQUE, INC.) | | | | | |
| Example 2 | Citric acid (anhydrous) (Nacalai Standard Class 1, | 100 g/L | 100 | 95 | 90 | 90 |
| | produced by NACALAI TESQUE, INC.) | | | | | |
| Example 3 | Citric acid (anhydrous) (Nacalai Standard Class 1, | 18 g/L | 85 | 84 | 83 | 82 |
| | produced by NACALAI TESQUE, INC.) | | | | | |
| Example 4 | DL malic acid (Nacalai Standard Class 1, produced | 30 g/L | 73 | 71 | 70 | 70 |
| | by NACALAI TESQUE, INC.) | | | | | |
| Example 5 | L-(+)-tartaric acid (Nacalai Standard Class 1, | 30 g/L | 72 | 70 | 63 | 60 |
| | produced by NACALAI TESQUE, INC.) | | | | | |
| Example 6 | Lactic acid (Nacalai Standard Class 1, produced by | 30 g/L | 75 | 70 | 65 | 60 |
| | NACALAI TESQUE, INC.) | | | | | |
| Comparative | — | — | 15 | 16 | 15 | 15 |
| Example 1 | | | | | | |
| Comparative | Adipic acid (Nacalai Standard Class 1, produced by | 30 g/L | 19 | 17 | 16 | 16 |
| Example 2 | NACALAI TESQUE, INC.) | | | | | |
| Comparative | Malonic acid (Nacalai Standard Class 1, produced by | 30 g/L | 21 | 18 | 17 | 15 |
| Example 3 | NACALAI TESQUE, INC.) | | | | | |
| Comparative | Polyacrylic acid resin (Aqualic HL415, solid content | 40 g/L | 100 | 24 | 20 | 15 |
| Example 4 | 45%, produced by Nippon Shokubai Co., Ltd.) | | | | | |
| Comparative | — | — | 24 | 24 | 25 | 23 |
| Example 5 | | | | | | |
| Comparative | Citric acid (anhydrous) (Nacalai Standard Class 1, | 100 g/L | 100 | 37 | 30 | 29 |
| Example 6 | produced by NACALAI TESQUE, INC.) | | | | | |

TABLE 2

| | | | | | | | Antimicrobial | Water | |
|---|---|---|---|---|---|---|---|---|---|
| | | | | | | | capability | absorb- | Water |
| | | | Ammonia deodorizing capability (%) | | | | After industrial | ability | repel- |
| | | | | After 10 | After 50 | After | washing | After 20 | lency |
| | | Concen- | Before | times of | times of | industrial | (Klebsiella | times of | Before |
| | Function-imparting agent | tration | washing | washing | washing | washing | pneumoniae) | washing | washing |
| Example 7 | Citric acid (anhydrous) | 18 g/L | 100 | 75 | 70 | 70 | 2.8 | — | — |
| | (Nacalai Standard Class 1, | | | | | | | | |
| | produced by NACALAI | | | | | | | | |
| | TESQUE, INC.) | | | | | | | | |
| | MR-T100 (produced by | 15 g/L | | | | | | | |
| | Osaka Kasei Co., Ltd.) | | | | | | | | |
| Example 8 | (First processing) MR-T100 | 15 g/L | 93 | 85 | 80 | 73 | 1.8 | — | — |
| | (produced by Osaka Kasei | | | | | | | | |
| | Co., Ltd.) | | | | | | | | |
| | (Second processing) Citric acid | 18 g/L | | | | | | | |
| | (anhydrous) (Nacalai | | | | | | | | |
| | Standard Class 1, produced by | | | | | | | | |
| | NACALAI TESQUE, INC.) | | | | | | | | |
| Example 9 | (First processing) MT-SS21 | 6% owf | 100 | 73 | 70 | 65 | — | 1 second or | — |
| | (produced by Matsumoto | | | | | | | less | |
| | Yushi-Seiyaku Co., Ltd.) | | | | | | | | |
| | (Second processing) Citric | 18 g/L | | | | | | | |
| | acid (anhydrous) (Nacalai | | | | | | | | |
| | Standard Class 1, produced | | | | | | | | |
| | by NACALAI TESQUE, INC.) | | | | | | | | |
| Example 10 | (First processing) MT-SS21 | 6% owf | 93 | 80 | 75 | 75 | — | 1 second or | — |
| | (produced by Matsumoto | | | | | | | less | |
| | Yushi-Seiyaku Co., Ltd.) | | | | | | | | |
| | (Second processing) Citric | 18 g/L | | | | | | | |
| | acid (anhydrous) (Nacalai | | | | | | | | |
| | Standard Class 1, produced | | | | | | | | |
| | by NACALAI TESQUE, INC.) | | | | | | | | |
| Example 11 | (First processing) Citric | 18 g/L | 100 | 83 | 75 | 70 | — | 1 second or | — |
| | acid (anhydrous) (Nacalai | | | | | | | less | |
| | Standard Class 1, produced | | | | | | | | |
| | by NACALAI TESQUE, INC.) | | | | | | | | |
| | (Second processing) SR1800 | 60 g/L | | | | | | | |
| | (water absorbing agent, | | | | | | | | |
| | produced by Takamatsu Fat & | | | | | | | | |
| | Oil Co., Ltd.) | | | | | | | | |
| | SR-CA-1 (catalyst for water | 6 g/L | | | | | | | |
| | absorbing agent, produced | | | | | | | | |
| | by Takamatsu Fat & Oil Co., | | | | | | | | |
| | Ltd.) | | | | | | | | |
| Example 12 | (First processing) Citric | 18 g/L | 90 | 85 | 75 | 70 | — | 1 second or | — |
| | acid (anhydrous) (Nacalai | | | | | | | less | |
| | Standard Class 1, produced | | | | | | | | |
| | by NACALAI TESQUE, INC.) | | | | | | | | |
| | (Second processing) SR1800 | 60 g/L | | | | | | | |
| | (water absorbing agent, | | | | | | | | |
| | produced by Takamatsu Fat & | | | | | | | | |
| | Oil Co., Ltd.) | | | | | | | | |
| | SR-CA-1 (catalyst for water | 6 g/L | | | | | | | |
| | absorbing agent, produced by | | | | | | | | |
| | Takamatsu Fat & Oil Co., Ltd.) | | | | | | | | |
| Example 13 | Citric acid (anhydrous) | 18 g/L | 100 | 75 | 70 | 70 | — | 1 second or | — |
| | (Nacalai Standard Class 1, | | | | | | | less | |
| | produced by NAKALAI | | | | | | | | |
| | TESQUE, INC.) | | | | | | | | |
| | SR1800 (water absorbing | 60 g/L | | | | | | | |
| | agent, produced by | | | | | | | | |
| | Takamatsu Fat & Oil Co., Ltd.) | | | | | | | | |
| | SR-CA-1 (catalyst for water | 6 g/L | | | | | | | |
| | absorbing agent, | | | | | | | | |
| | produced by Takamatsu Fat | | | | | | | | |
| | & Oil Co., Ltd.) | | | | | | | | |
| Example 14 | Citric acid (anhydrous) | 18 g/L | 95 | 71 | 70 | 70 | — | 1 second or | — |
| | (Nacalai Standard Class 1, | | | | | | | less | |
| | produced by NAKALAI | | | | | | | | |
| | TESQUE, INC.) | | | | | | | | |
| | SR1800 (water absorbing | 60 g/L | | | | | | | |
| | agent, produced by | | | | | | | | |
| | Takamatsu Fat & Oil Co., Ltd.) | | | | | | | | |
| | SR-CA-1 (catalyst for | 6 g/L | | | | | | | |
| | water absorbing agent, | | | | | | | | |
| | produced by Takamatsu | | | | | | | | |
| | Fat & Oil Co., Ltd.) | | | | | | | | |
| Example 15 | Citric acid (anhydrous) | 18 g/L | 85 | 70 | 65 | 60 | — | — | Class 4 |
| | (Nacalai Standard Class 1, | | | | | | | | |
| | produced by NACALAI | | | | | | | | |
| | TESQUE, INC.) | | | | | | | | |
| | FX860 (Fluorine-based water | 60 g/L | | | | | | | |
| | repellent produced by | | | | | | | | |
| | K.K. Kyokenkasei) | | | | | | | | |
| | Beckamin M-3 (triazine | 3 g/L | | | | | | | |
| | ring-containing compound, | | | | | | | | |
| | produced by Dainippon Ink | | | | | | | | |
| | and Chemicals, Inc.) | | | | | | | | |
| | Beckamin ACX (catalyst, | 1 g/L | | | | | | | |
| | produced by Dainippon Ink | | | | | | | | |
| | and Chemicals, Inc.) | | | | | | | | |
| Comparative | ELENITE 139 (produced by | 10 g/L | 30 | 25 | 25 | 23 | — | — | — |
| Example 7 | Takamatsu Fat & Oil Co., Ltd.) | | | | | | | | |

### INDUSTRIAL APPLICABILITY

Our poiyester-based fiber structure has all of high deodorizing capacity, advanced deodorizing capability excellent in washing durability, and good texture, and the polyester-based fiber structure can be used widely for general clothes and industrial materials requiring deodorizing capability and washing durability.

Further, if antimicrobial treatment, water-absorption treatment and water-repellency treatment are performed together, the polyester-based fiber structure can have the respective functions in combination, to be provided as polyester-based fiber structure having multiple functions.

1. A deodorizing fiber structure in which a material composed of a hydroxy acid derivative is secured to a polyester-based fiber structure.

2. The deodorizing fiber structure according to claim 1, wherein the material composed of said hydroxy acid derivative is a material composed of any one of the monomer, polymer or copolymer of the hydroxy acid derivative.

3. The deodorizing fiber structures according to claim 1, wherein the material composed of said hydroxy acid derivative is a derivative of at least one compound selected from citric acid, malic acid and tartaric acid.

4. The deodorizing fiber structure, according to claim 1, wherein the material composed of said hydroxy acid derivative is a derivative of citric acid.

5. The deodorizing fiber structure according to claim 1, wherein said polyester-based fiber structure contains a pyridine-based antimicrobial agent.

6. The deodorizing fiber structure, according to claim 1, wherein a water absorbing agent is deposited on said polyester-based fiber structure.

7. The deodorizing fiber structure according to claim 6, wherein said water absorbing agent is a hydrophilic polyester-base resin.

8. The deodorizing fiber structure according to claim 1. wherein a water repellent is deposited on said polyester-based fiber structure.

9. A method for producing a fiber structure comprising the steps of immersing a polyester-based fiber structure in a hydroxy acid aqueous solution, subsequently drying, and then beat-treating.
